(19) United States
(12) Patent Application Publication
(10) Pub. No.: US 2006/0024787 A1
(43) Pub. Date: Feb. 2, 2006

(54) Chimp trace amine associated receptors and polypeptides

(76) Inventors: Martin Ebeling, Grenzach-Wyhlen (DE); Marius Hoener, Basel (CH); Lothar Lindemann, Basel (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/174,751

(22) Filed: Jul. 5, 2005

(30) Foreign Application Priority Data
Jul. 30, 2004 (EP) 04103673.2

(51) Int. Cl. G01N 33/53; C07H 21/04; C12P 21/06; C07K 14/705

(52) U.S. Cl. 435/69.1; 435/320.1; 435/325; 435/7.1; 530/350; 536/23.5

(57) ABSTRACT

The present invention provides a fingerprint sequence which is specific and selective for chimp trace amine associated receptors (TAAR) forming a subfamily of G protein coupled receptors. The invention also provides the novel chimp polypeptides identified as members of this family, nucleic acids encoding said polypeptides, and vectors and host cells comprising the novel family members. In addition, the invention provides methods of identifying chimp TAARs.

### FIELD OF THE INVENTION

The present invention provides a fingerprint sequence which is specific and selective for chimp trace amine associated receptors (TAAR) forming a subfamily of G protein coupled receptors. The invention also provides the polypeptides identified as members of this family, nucleic acids encoding said polypeptides, and vectors, host cells and non-human animals comprising the novel family members. In addition, the invention provides methods of identifying TAARs.

### BACKGROUND OF THE INVENTION

Trace amines (TA) are endogenous compounds structurally related to biogenic amines and are found in the mammalian nervous system in trace amounts. TAs are stored in nerve terminals and released together with classical biogenic amines. To date there is no evidence for the existence of synapses using TAs as their exclusive transmitter. Recently, receptors specifically binding trace amines were reported by Borowski et al (Trace amines: identification of a family of mammalian G protein-coupled receptors. Proc Natl Acad Sci USA (2001) 98(16):8966-71) and Bunzow et al (Amphetamine, 3,4-methylenedioxymethamphetamine, lysergic acid diethylamide, and metabolites of the catecholamine neurotransmitters are agonists of a rat trace amine receptor. Mol Pharmacol. 2001, 60(6):1181-8.). These receptors apparently represent a novel GPCR-family.

The dysregulation of trace amines were linked to various psychiatric disorders like depression (Sandler M. et al., Decreased cerebrospinal fluid concentration of free phenylacetic acid in depressive illness. Clin Chim Acta. 1979, 93(1):169-71; Davis B A and Boulton A A. The trace amines and their acidic metabolites in depression—an overview. Prog Neuropsychopharmacol Biol Psychiatry. 1994, 18(1):17-45.), schizophrenia (Potkin S G et al., Phenylethylamine in paranoid chronic schizophrenia. Science. 1979, 206(4417):470-1; Sandler M and Reynolds G P. Does phenylethylamine cause schizophrenia? Lancet. 1976, 1(7950):70-1.) and bipolar disorder (Boulton A A.: Some aspects of basic psychopharmacology: the trace amines. Prog Neuropsychopharmacol Biol Psychiatry. 1982; 6(4-6):563-70; Sabelli H C et al., Clinical studies on the phenylethylamine hypothesis of affective disorder: urine and blood phenylacetic acid and phenylalanine dietary supplements. J Clin Psychiatry. 1986 February; 47(2):66-70.). There has also been made a link between dysregulation of trace amines and attention-deficit hyperactivity disorder (Baker et al., Phenylethylaminergic mechanisms in attention-deficit disorder. Biol Phychiatry. 1991, 29(1):15-22), Parkinson's disease (Heller B and Fischer E., Diminution of phenethylamine in the urine of Parkinson patients. Arzneimittelforschung. 1973, 23(6):884-6.), migraine (D'Andrea G. et al., Elusive amines and primary headaches: historical background and prospectives. Neurol Sci. 2003, 24 Suppl 2:S65-7; D'Andrea, G. et al., Elevated levels of circulating trace amines in primary headaches. Neurology. 2004, 62(10):1701-1705.) and eating disorders (Wolf M E and Mosnaim A D. Phenylethylamine in neuropsychiatric disorders. Gen Pharmacol. 1983, 14(4):385-90; Branchek T A and Blackburn T P. Trace amine receptors as targets for novel therapeutics: legend myth and tact. Curr. Opin Pharmacol. 2003. 3(1):90-97.) such as i.e. obesity and anorexia as suggested by the very high structural similarity between PEA and amphetamine, which is considered the strongest anorexic compound known to date. (Samanin R, and Garattini S.: Neurochemical mechanism of action of anorectic drugs. Pharmacol Toxicol. 1993 August; 73(2):63-8; Popplewell D A et al., A behavioural and pharmacological examination of phenylethylamine-induced anorexia and hyperactivity-comparisons with amphetamine. Pharmacol Biochem Behav. 1986 October; 25(4):711-6.).

Therefore, there is a broad interest to increase the knowledge about trace amine receptors, especially to identify further trace amine receptors. However, examination of the literature and public database entries revealed inconsistencies in the naming of the TA receptors, e.g. the human receptor GPR102 (Lee et al., Discovery and mapping of ten novel G protein-coupled receptor genes. Gene. 2001, 275(1):83-91.) is also referred to as TA5 (Borowski et al Trace amines: identification of a family of mammalian G protein-coupled receptors. Proc Natl Acad Sci USA (2001) 98(16):8966-71)), and human 5-HT4Ψ (Liu et al., A serotonin-4 receptor-like pseudogene in humans. Brain Res Mol Brain Res. 1998, 53(1-2):98-103.) has also been named TA2Ψ (Borowski et al., Trace amines: identification of a family of mammalian G protein-coupled receptors. Proc Natl Acad Sci USA (2001) 98(16):8966-71). In addition, GPR57 (Lee et al., Cloning and characterization of additional members of the G protein-coupled receptor family. Biochim Biophys Acta. 2000, 1490(3):311-23.), GPR58 (Lee et al., Cloning and characterization of additional members of the G protein-coupled receptor family. Biochim Biophys Acta. 2000, 1490(3):311-23.) and PNR (Zeng et al., Cloning of a putative human neurotransmitter receptor expressed in skeletal muscle and brain. Biochem Biophys Res Commun. 1998, 242(3):575-8.) were so far not generally recognized as TA receptor. This inconsistencies cause confusion and uncertainty. Therefore, there is a strong need for a clear definition of this receptor family.

### SUMMARY OF THE INVENTION

The present invention provides a fingerprint sequence which is specific and selective for chimp trace amine associated receptors (TAAR) forming a subfamily of G protein coupled receptors. The invention also provides the polypeptides identified as members of this family, nucleic acids encoding said polypeptides, and vectors, host cells and non-human animals comprising the novel family members. In addition, the invention provides methods of identifying TAARs.

In order to resolve any ambiguity of the current naming a novel, uniform nomenclature is proposed with this invention which refers to these receptors as Trace Amine Associated Receptors (TAARs). This novel nomenclature reflects the finding that at least some TAARs do not respond to TAs at all, hence the term “associated”. It covers all receptors of this GPCR family and is compatible with the different number of receptor genes in different species. The novel nomenclature is strictly based on the sequential order of the receptor genes on the respective human chromosome as well as a detailed phylogenetic analysis (FIG. 3) of the receptor genes across different species. The nomenclature adheres to the following rules:

Any two (or three) genes that are orthologues, i.e. that were generated through a speciation event, should be labeled with the same number. Vice versa, two genes must not have the same number if they are not orthologous.

Genes that are paralogues, i.e. that were generated through a gene duplication event within the lineage of one species, should be distinguished by a letter suffix.

Examples:
Genes hTAAR 5, rTAAR 5, mTAAR 5 are all orthologues.
Genes mTAAR 8a, b, c in mouse are paralogues.
Genes mTAAR 8a, b, c are all orthologues to hTAAR 8.

In addition, a comparison of the chimp TAAR proteins with the human equivalents may give indications to the functions of the human proteins. TAAR proteins, which are functional in both, human and chimpanzee, probably play an important physiologically role. On the other hand, TAAR proteins which are only functional in human but not in chimpanzee may account for the specific differences between human and chimpanzee.

The present invention provides novel chimp polypeptides identified as TAAR family members, the use of said polypeptides as drug target, the polynucleotide sequences encoding said polypeptide, and vectors, host cells and non-human animals comprising said polynucleotides. Furthermore, the present invention pertains the fingerprint motif specific and selective for the TAAR family and the use of it.

### FIGURES

FIG. 1 shows an alignment of all functional chimp TAARs. Amino acid residues conserved in all chimp, human, rat and mouse TAARs are highlighted by black shading. The characteristic chimp TAAR fingerprint motif is located in TM VII.

FIG. 2 shows schematic pseudogene ptTAAR2Ψ. Restriction sites are given above the diagram.

FIG. 3 shows the “repair” of the pseudogene hTAAR 3 (old name GPR57Ψ): The two bps CC in position 133-134 of the fixed ORF were added, which repairs the otherwise early stop codon TCA.

FIG. 4 shows the ligand pocket vectors of the functional chimp TAARs. The differences to the human orthologues are indicated by grey boxes.

FIG. 5 shows an alignment of human and chimp TAAR1.

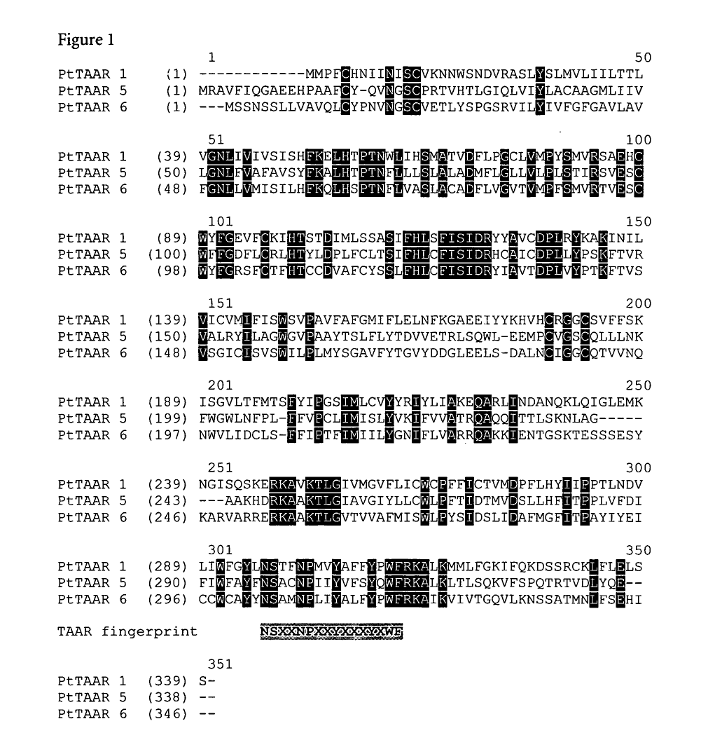

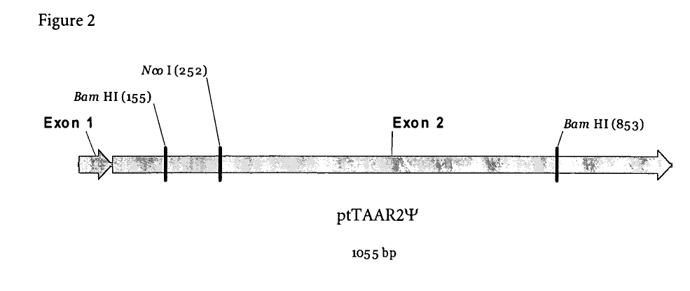

### GENERAL DEFINITIONS

As used herein, the singular form of any term also encompasses the plural form and vice-versa.

The term “gene” as used herein refers to any segment of DNA associated with a biological function. A gene is an ordered sequence of nucleotides located in a particular position on a particular chromosome that encodes a specific functional product.

The term “pseudogene” as used herein relates to an inactive gene which may have evolved by mutation events from an active ancestor gene. Inactive means that the gene is not translated to functional polypeptide.

The term “polypeptide sequence” (e.g., a protein, polypeptide, peptide, etc.) as used herein relates to a polymer of amino acids comprising naturally occurring amino acids.

The term “isolated” means altered “by the hand of man” from the natural state. If an “isolated” composition or substance occurs in nature, it has been changed or removed from its original environment, or both. For example, a polynucleotide or a polypeptide naturally present in a living animal is not “isolated”, but the same polynucleotide or polypeptide separated form the coexisting materials of its natural state is “isolated” as the term is employed herein.

The term “recombinant” when used with reference, e.g. to a polynucleotide or polypeptide typically indicates that the polynucleotide or polypeptide has been modified by the introduction of a heterologous (or foreign) nucleic acid of the alteration of a native nucleic acid, or that the protein or polypeptide has been modified by the introduction of a heterologous amino acid.

The term “polynucleotide sequence” (e.g., a nucleic acid, polynucleotide, oligonucleotide, etc.) as used herein relates to a polymer of nucleotides comprising nucleotides A, C, T, U, G.

The term “polypeptide of the invention” (polypeptide A) relates to one or more of the novel polypeptides provided in the present invention, i.e. ptTAAR1 to ptTAAR9.

### DETAILED DESCRIPTION OF THE INVENTION

The present invention provides novel chimp polypeptides identified as TAAR family members, the use of said polypeptides as drug target, the polynucleotide sequences encoding said polypeptide, and vectors, host cells and non-human animals comprising said polynucleotides. Furthermore, the present invention pertains the fingerprint motif specific and selective for the TAAR family and the use of it.

The present invention provides fingerprint motif comprising the sequence NSXXNPXXYXXXYXWF (SEQ. ID NO: 1), wherein X is any natural occurring amino acid and wherein the amino acid abbreviations N,S,P,Y,W and F are known to one of ordinary skill in the art as representing asparagine, serine, proline, tyrosine, tryptophan and phenylalanine respectively. The term “fingerprint”, “fingerprint motif” or “fingerprint sequence” as used herein relates to an amino acid sequence which is specific and selective for the chimp GPCR subfamily TAAR. Preferably, the fingerprint motif is specific for functional TAARs. The tryptophan (W) residue in the context of the fingerprint motif is found exclusively in TAARs and not in any other known GPCR; rather, the corresponding sequence position is almost invariably occupied by polar or even charged amino acids in other GPCRs. The fingerprint motif may be used for identifying TAARs, preferably for identifying functional TAARs.

The present invention also provides a method of identifying chimp TAARs using the fingerprint sequence (SEQ. ID NO: 1) by comparing the identity of said sequence with a polypeptide sequence. To be identified as a member of the chimp TAARs family a polypeptide may comprise a sequence having 100% identity with the fingerprint sequence.

Furthermore, the present invention pertains a method of identifying TAARs of other species, preferably of mammalians, using the fingerprint sequence (SEQ. ID NO: 1). To be identified as a member of the mammalian TAARs family a polypeptide may comprise a sequence having at least 75% identity with the fingerprint sequence, preferably more than 87% identity, more preferably 100% identity.

The fingerprint sequence may be used as a “query sequence” to perform a search against sequence databases to, for example to identify TAAR family members. Such searches can be performed using a pattern recognition program as for example fuzzpro of EMBOSS (Rice et al., EMBOSS: the European Molecular Biology Open Software Suite. Trends in Genetics, 2000, 16(6):276-277). A search for chimp TAAR may be performed with fuzzpro, NSXXNPXXYXXXYXWF as the required sequence, number of mismatches=0, database=swissprot (release 43).

The chimp TAAR family contains eight members (ptTAAR1 to ptTAAR 9) wherein five of them are pseudogenes: ptTAAR2 Ψ, ptTAAR3 Ψ, ptTAAR4 Ψ, ptTAAR8 Ψ, ptTAAR9 Ψ. All genes encoding the chimp TAAR family members are located on chimp chromosome 5. With the exception of ptTAAR 2, which is encoded by two exons, the coding sequences of the chimp TAAR genes are located in a single exon.

The present invention provides an isolated or recombinant polypeptide comprising sequence SEQ. ID NO: 3. Said polypeptide was identified as trace amine associated receptor (TAAR) and was named ptTAAR1.

The present invention further pertains the polynucleotide sequence comprising SEQ. ID NO: 2 that encodes the polypeptide ptTAAR1.

The present invention provides further an isolated or recombinant polypeptide comprising sequence SEQ. ID NO: 14. Said polypeptide was identified as trace amine associated receptor and was named ptTAAR5.

The present invention also pertains the polynucleotide sequence comprising SEQ. ID NO: 13 that encodes the polypeptide ptTAAR5.

The present invention provides further an isolated or recombinant polypeptide comprising sequence SEQ. ID NO: 16. Said polypeptide was identified as trace amine associated receptor and was named ptTAAR6.

The present invention also pertains the polynucleotide sequence comprising SEQ. ID NO: 15 that encodes the polypeptide ptTAAR6.

The present invention also provides the nucleotide sequences of ptTAAR2Ψ (SEQ. ID NO: 4).

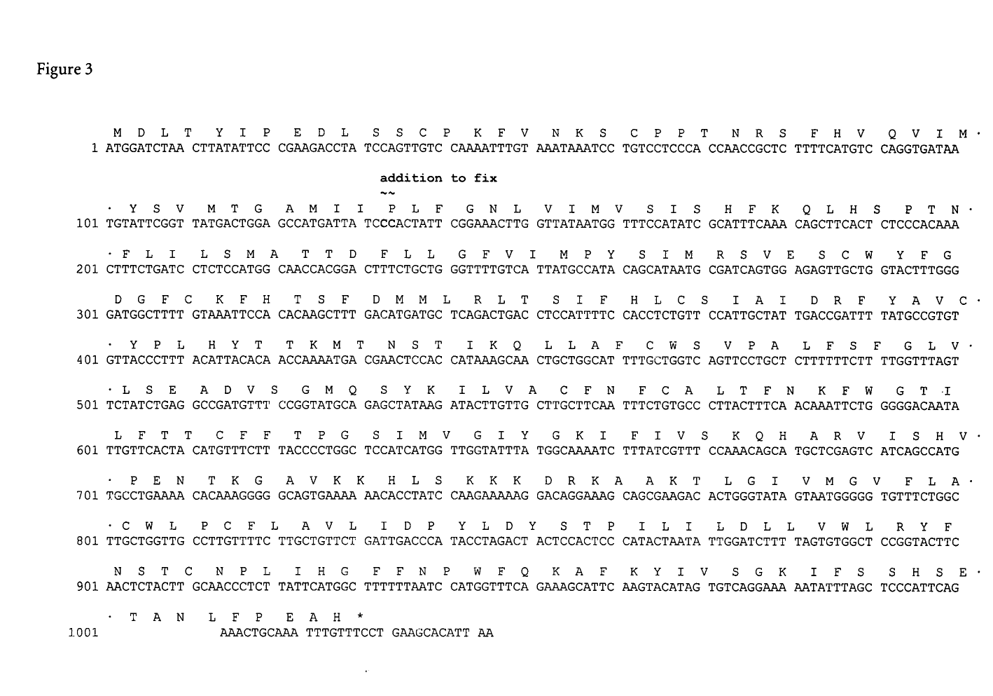

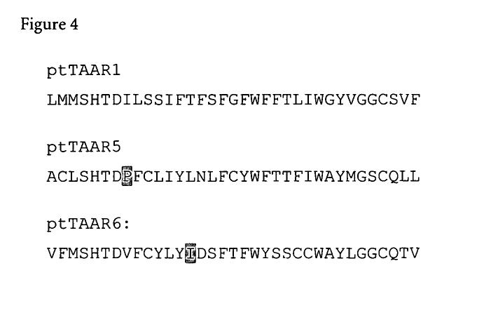

Furthermore, the present invention provides the repaired nucleotide sequences of ptTAAR2Ψ (ptTAAR2Ψf, SEQ. ID NO: 5). This invention further provides the polynucleotide sequences encoded by the fixed nucleotide sequence ptTAAR2Ψf (SEQ. ID NO: 6). The term “repaired gene” or “fixed gene” as used herein relates to a pseudogene whose sequence was altered in a way that the gene becomes active. The polynucleotide sequence of i.e. hTAAR3 is inactive due to a mutation that caused an early stop codon. The sequence was repaired by insertion of two nucleotides at position 133-134 (see FIG. 3).

The present invention also provides the nucleotide sequences of ptTAAR3Ψ (SEQ. ID NO: 7).

Furthermore, the present invention provides the repaired nucleotide sequences of ptTAAR3Ψ (ptTAAR3Ψf, SEQ. ID NO: 8). This invention further provides the polynucleotide sequences encoded by the fixed nucleotide sequence ptTAAR3Ψf (SEQ. ID NO: 9).

The present invention also provides the nucleotide sequences of ptTAAR4Ψ (SEQ. ID NO: 10). Furthermore, the present invention provides the repaired nucleotide sequences of ptTAAR4Ψ (ptTAAR4Ψf, SEQ. ID NO: 11). This invention further provides the polynucleotide sequences encoded by the fixed nucleotide sequence ptTAAR4Ψf (SEQ. ID NO: 12).

The present invention also provides the nucleotide sequences of ptTAAR8Ψ (SEQ. ID NO: 17).

Furthermore, the present invention provides the repaired nucleotide sequences of ptTAAR8Ψ (ptTAAR8Ψf, SEQ. ID NO: 18). This invention further provides the polynucleotide sequences encoded by the fixed nucleotide sequence ptTAAR8Ψf (SEQ. ID NO: 19).

The present invention also provides the nucleotide sequences of ptTAAR9Ψ (SEQ. ID NO: 20). Furthermore, the present invention provides the repaired nucleotide sequences of ptTAAR9Ψ (ptTAAR9Ψf, SEQ. ID NO: 21). This invention further provides the polynucleotide sequences encoded by the fixed nucleotide sequence ptTAAR9Ψf (SEQ. ID NO: 22).

Furthermore, the present invention also provides isolated or recombinant chimp polypeptides comprising an amino acid sequence which comprise the fingerprint sequence (SEQ. ID NO: 1).

In a further aspect the invention relates to the use of the polypeptides of the invention as drug target. Preferably, the polypeptides of the invention are used as a drug target for identifying compounds useful in depression therapy, in schizophrenia therapy, in migraine therapy, or in therapy of attention-deficit hyperactivity disorder or eating disorder as anorexia or obesity. The drug target may be suitable for the design, screening and development of pharmaceutically active compounds.

One embodiment of the invention relates to the polypeptide having a polypeptide sequence SEQ. ID NO: 3, 6, 9, 12, 14, 16, 19 or 22 as drug target. Preferably, the polypeptides of the invention are used as a drug target for identifying compounds useful in depression therapy, in schizophrenia therapy, in migraine therapy, or in therapy of attention-deficit hyperactivity disorder or eating disorder as anorexia or obesity.

Another embodiment of the invention relates to chimp receptors having an amino acid sequence which comprise the fingerprint sequence (SEQ. ID. NO: 1) as drug target. Preferably, these receptors are drug target for identifying compounds useful in depression therapy, in schizophrenia therapy, in migraine therapy, or in therapy of attention-deficit hyperactivity disorder or eating disorder as anorexia or obesity.

The present invention also pertain vectors comprising polynucleotides of the invention, host cells which are transduced with said vectors and the production of the polypeptides of the invention by recombinant techniques. Preferably, the vector comprises the polynucleotide comprising SEQ. ID NO: 2, SEQ. ID NO: 5, SEQ. ID NO: 8, SEQ. ID NO: 11, SEQ. ID NO: 13, SEQ. ID NO: 15, SEQ. ID NO: 18 or SEQ. ID NO: 21 and preferably, the host cells are transduced with said vector.

Host cells can be genetically engineered (i.e., transduced, transformed or transfected) to incorporate expression systems or portion thereof for polynucleotides of the present invention. Introduction of the vector into host cells can be effected by methods described in many standard laboratory manuals, such as Davis et al., Basic methods in molecular biology Elsevier, N.Y. (1986); Davis J M (ed.): Basic cell culture: a practical approach, sec. edition. Oxford University Press (2002); R. Ian Freshney: Culture of Animal Cells: A Manual of Basic Technique, fourth edition. John Wiley & Sons (Sd) 1999; and Sambrook et al., Molecular cloning: a laboratory manual, 2. Ed., Cold Spring Harbour Laboratory Press, Cold Spring Harbor, N.Y. (1989) (hereby incorporated by reference) and including methods such as, for example, calcium phosphate transfection, DEAE-dextran mediated transfection, transvectin, microinjection, cationic lipid-mediated transfection, electroporation, transduction or infection.

A host cell may be mammalian cell as HEK 293, CHO, COS, HeLa, neuronal, neuroendocrinal, neuroblastomal or glial cell lines like SH-SY5Y, PC12, HN-10 (Lee H J, Hammond D N, Large T H, Roback J D, Sim J A, Brown D A, Otten U H, Wainer B H.: Neuronal properties and trophic activities of immortalized hippocampal cells from embryonic and young adult mice. J Neurosci. 1990 June; 10(6):1779-87.), IMR-32, NB41A3, Neuro-2a, TE 671, primary neuronal or glia cells from mammals like rat or mouse, Xenopus oocytes, bacterial cells such as streptococci, staphylocooci, E. coli, Streptomyces and Bacillus subtilis cells; fungal cells such as Saccharomyces cerevisiae and Aspergillus cell; insect cells such as Drosophila S2 and Spodoptera Sf9 cells and plant cells.

A great variety of expression systems can be used. Such a system include, among others, chromosomal, episomal and virus-derived systems, i.e. vectors derived from bacterial plasmids, from bacteriophage, from transposons, from yeast episoms, from yeast chromosomal elements, from viruses such as baculovirus, papova viruses, such as SV40, vaccinia viruses, adenovirus, fowl pox virus, pseudorabies, retroviruses and vectors derived from combinations thereof, such as those derived from plasmids and bacteriophage genetic elements, such as cosmids and phagemids. The expression systems may contain control regions that regulate as well as engender expression. Generally, any system or vector suitable to maintain, propagate or express polynucleotides to produce a polypeptide in a host may be used. The appropriate nucleotide sequence may be inserted into an expression system by any of a variety of well-known and routine techniques, such as, for example, those set forth on Sambrook et al., Molecular cloning: a laboratory manual, 2. Ed., Cold Spring Harbour Laboratory Press, Cold Spring Harbour, N.Y. (1989) or Borowski et al Trace amines: identification of a family of mammalian G protein-coupled receptors. Proc Natl Acad Sci USA (2001) 98(16):8966-71), both of which are hereby incorporated by reference.

The present invention further provides a transgenic non-human animal comprising a polynucleotide encoding a polypeptide of the invention. Preferably, the transgenic non-human animal comprises a polynucleotide comprising (or selected from one or more of the group consisting of) SEQ. ID NO: 2, SEQ. ID NO: 5, SEQ. ID NO: 8, SEQ. ID NO: 11, SEQ. ID NO: 13, SEQ. ID NO: 15, SEQ. ID NO: 18 or SEQ. ID NO: 21.

The transgenic non-human animal may be any non-human animal known in the art. Preferably, the transgenic non-human animal is a mammal, more preferably the transgenic non-human animal is a rodent. Most preferably, the transgenic animal of the invention is a mouse.

Methods of producing a transgenic non-human animal are well known in the art such as for example those set forth on Hogan, B. C., F; Lacy, E, Manipulating the Mouse Embryo: A Laboratory Manual. 1986, New York: Cold Spring Harbor Laboratory Press; Hogan, B., et al., Manipulating the mouse embryo. 1994, Second Edition, Cold Spring Harbor Press, Cold Spring Harbor and Joyner, A. (ed.): Gene Targeting—A Practical Approach Second Edition. Practical Approach Series, Oxford University Press 1999, all of which are hereby incorporated by reference.

The polypeptides of the invention may be employed in a screening process for compounds (candidate compounds) which bind the receptor and which activate (agonists) or inhibit (antagonists) activation of the receptor polypeptide of the present invention (polypeptide A) or compounds which modulate receptor function e.g. by acting on receptor trafficking. Candidate compounds are thus any compound of interest that would bind (to) the receptor and/or would modulate (ie. activate (agonist compounds) or inhibit (antagonist compounds)) activation of the receptor polypeptide at the invention (polypeptide A). A test candidate compound, or test compound, is any compound of interest that one skilled in the art may wish to test to screen and identify if said compound would bind (to) the receptor and/or would modulate (ie. activate (agonist compounds) or inhibit (antagonist compounds)) activation of the receptor polypeptide at the invention (polypeptide A).

The present invention provides a method for identifying candidate compounds which bind to the polypeptide of the invention comprising:

a) contacting the polypeptide of the invention with a test candidate compound and

b) determining whether said compound binds to the polypeptide of the invention.

The invention also provides a method for identifying candidate compounds which modulate, or in other words, have a stimulatory or inhibitory effect on the biological activity of polypeptide of the invention or its expression comprising

a) contacting the polypeptide of the invention with a test candidate compound and

b) determining if said compound has modulated the function or activity of the polypeptide of the invention.

The invention thus also provides a method for screening and identifying candidate compounds useful in a selected therapy comprising: contacting polypeptide A with the test candidate compound (test compound); determining whether said compound binds to the polypeptide A; and determining whether said binding modulates (activates or inhibits) the biological activity of the polypeptide A, wherein the modulation (activation or inhibition) of the biological activity of the polypeptide A by said binding identifies whether said test candidate compound (test compound) is useful in said therapy. The selected therapy is preferably selected from one of the group consisting of depression therapy, schizophrenia therapy, migraine therapy, eating disorder therapy and attention-deficit hyperactivity disorder therapy.

In general, such screening procedures as described above involve producing appropriate cells which express the receptor polypeptide of the present invention on the surface thereof, however, the receptor expressed in appropriate cells may also be localized intracellularly. Such cells include i.e. cells from mammals, Xenopus, yeast, Drosophila or E. coli. Cells expressing the receptor (or cell membrane containing the expressed receptor) are then contacted with a test compound to measure binding, or stimulation or inhibition of a functional response.

One screening technique includes the use of cells which express receptor of this invention (for example, transfected CHO cells or HEK293) in a system which measures extracellular pH or intracellular calcium changes caused by receptor activation. In this technique, compounds may be contacted with cells expressing the receptor polypeptide of the present invention. A second messenger response, e.g., signal transduction, pH changes, PI hydrolyse, GTP-γ-[³⁵S] release or changes in calcium level, is then measured to determine whether the potential compound activates or inhibits the receptor.

Another method involves screening for receptor inhibitors by determining inhibition or stimulation of receptor-mediated cAMP and/or adenylate cyclase accumulation. Such a method involves transfecting a eukaryotic cell with the receptor of this invention to express the receptor on the cell surface. The cell is then exposed to potential antagonists in the presence of the receptor of this invention. The amount of cAMP accumulation is then measured. If the potential antagonist binds the receptor, and thus inhibits receptor binding, the levels of receptor-mediated cAMP, or adenylate cyclase, activity will be reduced or increased.

Another method for detecting agonists or antagonists for the receptor of the present invention is the yeast based technology as described in U.S. Pat. No. 5,482,835, hereby incorporated by reference.

The assays may simply test binding of a candidate compound wherein adherence to the cells bearing the receptor is detected by means of a label directly or indirectly associated with the candidate compound or in an assay involving competition with a labeled competitor. Further, these assays may test whether the candidate compound results in a signal generated by activation of the receptor, using detection systems appropriate to the cells bearing the receptor at their surfaces. Inhibitors of activation are generally assayed in the presence of a known agonist and the effect on activation by the agonist by the presence of the candidate compound is observed.

Standard methods for conducting such screening assays are well understood in the art.

Examples of potential polypeptide of the invention antagonists include antibodies or, in some cases, oligonucleotides or proteins which are closely related to the ligand of the polypeptide of the invention, e.g., a fragment of the ligand, or small molecular weight molecules such as e.g. metabolites of neurotransmitters or of amino acids, which bind to the receptor but do not elicit a response, so that the activity of the receptor is prevented.

In addition, the present invention provides methods, polynucleotides, polypeptides, nucleic acid primer and uses substantially as herein before described especially with reference to the following examples.

Having now generally described this invention, the same will become better understood by reference to the specific examples, which are included herein for purpose of illustration only and are not intended to be limiting unless otherwise specified, in connection with the following figures.

### EXAMPLES

Commercially available reagents referred to in the examples were used according to manufacturer's instructions unless otherwise indicated.

Identification of TAAR Family Members

The TAAR genes were identified by comparison of the previously published TAAR receptor genes with the chimp genomic sequence information available from Genbank (see e.g., Table 1) employing standard algorithms like BLAST. Where the chimp genomic sequence information was not available, the information was extrapolated from the human genome sequences available from the Genbank.

TABLE 1
members of the chimp TAAR family.

| Name | | | SEQ. ID NO | | |
|---|---|---|---|---|---|
| Generic | Chimpanzee | bp | nt. | nt. fixed | aa. |
| ptTAAR 1 | Novel | 1020 | 2 | — | 3 |
| ptTAAR 2 | Novel ψ | 1055 | 4 | 5 | 6 |
| ptTAAR 3 | Novel ψ | 1030 | 7 | 8 | 9 |
| ptTAAR 4 | Novel ψ | 1049 | 10 | 11 | 12 |
| ptTAAR 5 | Novel | 1014 | 13 | — | 14 |
| ptTAAR 6 | Novel | 1038 | 15 | — | 16 |
| ptTAAR 8 | Novel ψ | 1027 | 17 | 18 | 19 |
| ptTAAR 9 | Novel ψ | 1048 | 20 | 21 | 22 |

pt = Pan troglodytes,
nt = nucleotide sequence,
nt fixed = repaired nucleotide sequence,
aa = amino acid sequence. The amino acid sequence of ptTAAR 2, ptTAAR3, ptTAAR4, ptTAAR8 and ptTAAR9 is the counterpart of the repaired nucleotide sequence.

Based on this sequence information the TAAR genes were amplified by means of PCR from chimp genomic DNA. Where not further specified, all procedures were carried out basically described in: Sambrook, J., Fritsch, E. F., und Maniatis, T. (1989). Molecular Cloning: A laboratory manual (New York: Cold Spring Harbor Laboratory Press) hereby incorporated by reference. All reagents were of highest available purity, and all solutions and reagents were sterilized prior to use unless otherwise stated.

For this purpose oligonucleotide primers (Table 2) were designed based on the TAAR coding sequences which had been derived from the genomic sequence information available at Genbank. The primers were designed such that the amplicons contain the entire open reading frames. The primer were designed using the VectorNTI software (Vector NTI version 9.0.0, Informax) following standard rules for PCR primers, mainly: a) Primers should have a length of 18-25 nt b) a G/C content of about 50% c) should not contain inverted repeats longer than 3 nt d) should carry an G or C at least at the 5′ end e) should have no simple repetitions of the same nts more than 3, and f) the annealing temperatures (TM) of primers used in the same PCR reaction should differ as little as possible (for details see for example: McPherson M. J., Hames B. D., Taylor G. R. (eds.): PCR 2—A Practical Approach. The practical approach series, Oxford University Press, 1995, ISBN: 0-19-963424-6). The annealing temperatures of the primer oligonucleotides were calculated based on rules described in: Breslauer K J, Frank R, Blocker H, Marky L A.: Predicting DNA duplex stability from the base sequence. Proc Natl Acad Sci USA. 1986 June; 83(11):3746-50 (These rules assume that the sequences are not symmetric and contain at least one G or C and have a length of minimally 8 nts). Oligonucleotides were ordered from Microsynth (Microsynth A G, Schützenstrasse 15, 9436 Balgach, Switzerland) in HPLC purified quality.

Due to the draft quality of the chimpanzee genome sequence release the sequence of some ptTAAR genes, or parts thereof, were not available. Therefore, some primers had to be designed based on the corresponding human genome sequences. In those instances and wherever possible, primers where placed outside of the coding regions (Table 2).

TABLE 2
Primer for cloning of ptTAARs.

| | | | | SEQ | |
|---|---|---|---|---|---|
| Receptor | Primer names | Sequence (5′ → 3′) | TM | ID NO | Comment |
| ptTAAR 1 | schTAR1_5_01 | atgatgcccttttgccac | 59.2 | 23 | |
| | schTAR1_3_01 | ctatgaactcaattccaaaaataattt | 57.4 | 24 | |
| ptTAAR 2 | schGPR58_5_01 | gaaacattcaattgctctgaatatg | 58.9 | 25 | |
| (exon II) | schGPR58_3_02 | tgcttcaatttattcatgcag | 56.3 | 26 | human |
| ptTAAR 2 | chGPR58_ex1_5_02 | ctaaggagcctgatctcaacc | 57.8 | 27 | |
| (exon I) | chGPR58_ex1_3_01 | gctctgtgtgatctccgttg | 59.2 | 28 | |
| ptTAAR 3 | schGPR57_5_02 | cagtgactcatcctcctgg | 56.1 | 29 | human |
| | schGPR57_3_02 | tctattcacttttgcaacagc | 55.4 | 30 | human |
| ptTAAR 4 | schTAR2_5_01 | atgatgaatttgcctgaccc | 59.5 | 31 | |
| | schTAR2_3_01 | ctaagcatgggcagaaaacag | 59.7 | 32 | |
| ptTAAR 5 | schPNR_5_01 | atgagagctgtcttcatccaag | 58.3 | 33 | |
| | schPNR_3_01 | tcattcttggtacaaatcaacag | 56.6 | 34 | |
| ptTAAR 6 | schTAR4_5_02 | cttctccatatgtaaataacagcg | 57.0 | 35 | |
| | schTAR4_3_02 | gtatcctgaacttcgtctatacaac | 55.2 | 36 | |
| ptTAAR 8 | schTAR5_5_01 | atgaccagcaatttttcctaac | 57.1 | 37 | human |
| | schTAR5_3_01 | ttattctgaaaataaactaatggctg | 57.2 | 38 | human |
| ptTAAR 9 | schTAR3_5_01 | atggtgaacaatttctccc | 54.3 | 39 | |
| | schTAR3_3_01 | ttaatctttctctacttcttcagaaaa | 55.7 | 40 | |

ptTAAR2: The two exons of this gene, predicted to encode GPR58 based upon the finding in the human gene, were cloned separately from genomic DNA; human: primers were designed based on the corresponding human genome sequences. (Tm: melting temperature in ° C. calculated according to Breslauer K J, Frank R, Blocker H, Marky L A.: Predicting DNA duplex stability from the base sequence. Proc Natl Acad Sci USA. 1986 June; 83(11):3746-50; 5 in the primer name indicates a 5′ primer (i.e. schTAR3_{—}5_{—}01) and 3 in the primer name indicates a 3′ primer).

The actual PCR reactions had a total volume of 50 μl and the following composition: Template equaling 5-100 ng genomic DNA (source specified below), 200 nM oligonucleotide primer, 1.5 mM MgCl₂ (Invitrogen), 200 mM dNTPs each (Invitrogen), 1× concentrated PCR reaction buffer (Invitrogen) and 5 U/reaction recombinant Taq DNA polymerase (Invitrogen). The PCR reactions were assembled under a sterile working bench with UV irradiated equipment, and a) preparation of template material, b) assembly of PCR reactions, c) running the PCR reactions and agarose gel electrophoresis, and d) plasmid preparation were each performed in separated rooms in order to avoid any possible cross contamination of reagents with PCR amplified material or plasmid DNA. The PCR reactions were assembled in 200 μl PCR cups (Eppendorf) including Taq DNA polymerase at room temperature (RT) and transferred to the thermocycler (Geneamp 9700 with gold block, Applied Biosystems) preheated to 95° C. The temperature profile was adjusted as follows:

| | |
|---|---|
| 95° C. | 2 min |

(95° C.: 30 sec, annealing temperature: 30 sec, 72° C.: extension time)×cycle number:

| | | |
|---|---|---|
| 72° C. | 5 | min |
| 4° C. | max. 6 | hrs |

annealing temperature: the Tm (melting temperature) of the oligonucleotide primer with the lower Tm value for the PCR reaction (calculated as specified above) −1° C. was used as annealing temperature. Example: 2 primers with Tm primer 1=55° C., Tm primer 2=57° C. [symbol] annealing temperature=54° C.

extension time was calculated by length of amplicon (in kb)×1 min.

cycle number: For each gene several PCR reactions were run in parallel with cycle numbers between 25-40. All PCR reactions were subsequently analyzed by agarose gel electrophoresis, and the PCR reaction with the lowest cycle number still producing a clearly visible PCR product of the correct size was used for subsequent cloning.

The PCR products were analyzed by agarose gel electrophoresis using 1% ultraPure agarose (GibcoBRL) made up in TAE buffer (Invitrogen). Agarose gels were run in PerfectBlue Horizontal Mini Electrophoresis Systems (Pequlab Biotechnologie GmbH, Erlangen, Germany) according to standard protocols (Sambrook et al., 1989). Agarose gels were stained with ethidium bromide, PCR products were visualized on a UV transilluminator (Syngene, Cambridge, UK), and the size of the PCR products was analyzed in comparison with the molecular weight standard 1 kb DNA ladder (Invitrogen). PCR products of the expected sizes were cut out from the gels with sterile scalpels (Bayha, Tuttlingen, Germany) and extracted from the agarose gel slices using the QIAquick Gel Extraction Kit (QIAGEN AG, Basel, Switzerland) following the instructions of the manufacturer.

The extracted PCR products were precipitated with a cold ethanol/sodium acetate precipitation (Sambrook et al., 1989), and DNA pellets were dissolved in a volume of 10 μl of 10 mM Tris/HCl pH 7.5. The PCR products in this solutions were subsequently cloned using the TOPO cloning kit for sequencing (Invitrogen; kits containing the vectors pCR4-TOPO and pCR2.1-TOPO were used) following the instructions of the manufacturer. Ligations were transformed into TOP10 chemically competent bacteria (included in the TOPO cloning kit for sequencing) following the instructions of the manufacturer and subsequently plated on LB agar plates containing 100 μg/ml ampicillin (Sigma, Division of FLUKA Chemie GmbH, Buchs, Switzerland) and incubated at 37° C. over night. The bacterial colonies were analyzed by a method called “miniprep PCR”: Colonies were picked with a sterile inoculation loop (Copan Diagnostic S.P.A., Brescia, Italy) and transferred to new LB agar plates containing 100 μg/ml ampicillin. The same inoculation loops were subsequently transferred individually into 50 μl 10 mM Tris/HCl pH 7.5 in 1.5 ml Eppendorf Cups (Eppendorf) in order to transfer the bacteria which had remained on the loop into the solution. This bacterial suspensions were subsequently heated to 95° C. for 5 min, and 1 μl pf the resulting bacterial lysates per 50 μl PCR reaction was used as template for PCR reactions with primers flanking the multiple cloning site of the plasmids pCR4-TOPO and pCR2.1-TOPO (T7 and M13 reverse, sequences: Primer “T7”: 5′-cgggatatcactcagcataat-3′, primer “M13 reverse”: 5′-caggaaacagctatgacc-3′). PCR reactions were assembled as described above and run with the following temperature profile:

| | |
|---|---|
| 95° C. | 2 min |

(95° C.: 30 sec, 50° C.: 30 sec, 72° C.: 1 min)×30 cycles

| | | |
|---|---|---|
| 72° C. | 5 | min |
| 4° C. | max. 6 | hrs. |

The PCR products were analyzed by agarose gel electrophoresis as described above. Bacterial colonies for which cloned DNA fragments of the expected sizes could be detected by the method outlined above were used for subsequent plasmid preparation.

For plasmid preparations bacterial colonies identified to carry inserts of the expected sizes by “miniprep PCR” were used to inoculate bacterial liquid cultures in LB medium containing 100 μg/ml ampicillin, and cultures were incubated on a horizontal shaker at 37° C. over night (Sambrook et al., 1989). Plasmids were isolated from the bacterial liquid cultures using the HiSpeed Plasmid Maxi Kit (QIAGEN AG, Basel, Switzerland) following the instructions of the manufacturer. Plasmid concentrations were determined by measuring the OD at 260 nm using an UV/VIS spectrophotometer ultrospec 3300 pro (Amersham Biosciences Europe GmbH, Otelfingen, Switzerland). The size of inserts in the isolated plasmids was analyzed by means of restriction digests using an restriction endonuclease i.e. EcoRI (New England Biolabs, products distributed in Switzerland by Bioconcept, Allschwil, Switzerland); EcoRI restriction sites are flanking the multiple cloning sites in both vectors pCR4-TOPO and pCR2.1-TOPO, therefore the cloned inserts can be released from the plasmids by EcoRI restriction digests. Of each plasmid 0.5 μg were digested with EcoRI in a total volume of 20 μl following the recommendations of the manufacturer. Restriction digests were analyzed by agarose gel electrophoresis as described above. Plasmids which turned out to carry inserts of the expected sizes as revealed by restriction analysis were subjected to DNA sequence analysis, carried out by an external company (Microsynth AG, Schützenstrasse 15, 9436 Balgach, Switzerland).

The DNA sequences of the cloned TAAR gene open reading frames, as revealed by DNA sequence analysis, were compared to either previously published sequences and/or to the sequence of the TAAR genes retrieved from the genome sequence information. Potential errors in the DNA sequence information which could have been introduced by the PCR reaction were eliminated by comparing the sequences of the two to three DNA plasmids which were cloned each from independent PCR reactions per TAAR gene: Since the probability of the same PCR error to occur at the very same position in independent PCR reactions is basically zero, the alignment of the independent sequences for each gene revealed the very correct DNA sequence.

Template material: chimpanzee (pan troglodytes) genomic DNA was purchased from Southwest National Primate Research Center (SNPRC, P.O. Box 760549; San Antonio, Tex., USA). The DNA concentration was determined by measuring the OD at 260 nm as described above and the DNA concentration was adjusted to 100 ng/μl. The DNA was stored in aliquots at 4° C.

Cell Culture

Subcloning: For expression of TAAR receptors in mammalian cell lines, DNA fragments carrying the entire TAAR open reading frames were subcloned from the pCR4-TOPO or pCR2.1-TOPO vectors (Invitrogen) to pIRES-NEO2 (Rees S et al., Bicistronic vector for the creation of stable mammalian cell lines that predisposes all antibiotic-resistant cells to express recombinant protein. Biotechniques. 1996 January; 20(1):102-4, 106, 108-10).

To this end, DNA fragments carrying the entire TAAR open reading frames were removed from the respective TOPO vectors by restriction digest with i.e. EcoRI and subsequent purification of the ˜1 kb fragments by agarose gel electrophoresis and gel extraction as described above. In parallel, the pIRES-NEO2 vector was linearized with EcoRI and dephosphorylated with shrimp alkaline phosphatase (Roche) following the instructions of the manufacturer. The DNA fragments carrying the entire TAAR open reading frames were ligated into the linearized pIRES-NEO2 vector using T4 DNA ligase (New England Biolabs) by mixing 30 ng of linearized pIRES-NEO2 vector, 100-300 ng of DNA fragments carrying the respective TAAR coding sequence, 1× ligation buffer (final concentration; provided with the enzyme as 10× concentrate) and 1 μl of T4 DNA ligase in a total volume of 20 μl. The reaction was proceeded at room temperature for 2-3 hrs, and the ligation was transformed into chemically competent TOP10 cells as described for the TOPO ligations. Plating of transformed bacteria and picking of bacterial clones was performed as described earlier for the PCR cloning of TAAR genes from genomic and cDNA. The picked bacterial clones were used to inoculate 5 ml liquid cultures in LB containing 100 μg/ml ampicillin. These liquid cultures were used for mini plasmid preparations using the QIAprep Miniprep Kit (QIAGEN), following the instructions of the manufacturer. The purified pIRES-NEO2 derived plasmid constructs were analyzed for the presence of inserts of the expected sizes by restriction analysis using EcoRI and agarose gel electrophoresis. Plasmids which were identified to carry inserts of the expected sizes were subjected to DNA sequence analysis carried out by Microsynth in order to make sure for the correct orientation and sequence of the inserts. Plasmids carrying the correct inserts in the correct orientation were used for expression of the respective TAARs in mammalian cell lines.

Cell culture: Basic cell culture handling and techniques used are described in Davis J M (ed.): Basic cell culture, sec. edition. Oxford University Press 2002, ISBN: 0199638535. TAARs were expressed in HEK cells (ATCC number: CRL-1573; described in Graham, F. L., Smiley, J., Russell, W. C., und Nairn, R. (1977). Characteristics of a human cell line transformed by DNA from human adenovirus type 5. Journal of General Virology 36, 59-74). The culture medium consisted of DMEM with Glutamax I, with sodium pyruvate, with pyridoxine, with 4500 mg/l glucose, Invitrogen Cat. # 31966-021; Penicillin/Streptomycin; fetal calf serum heat inactivated 10%. Cells were passaged at a ratio of 1:10-1:30 using Trypsin/EDTA (Invitrogen Cat. # 25300-062).

Comparison of Human and Chimpanzee TAARs

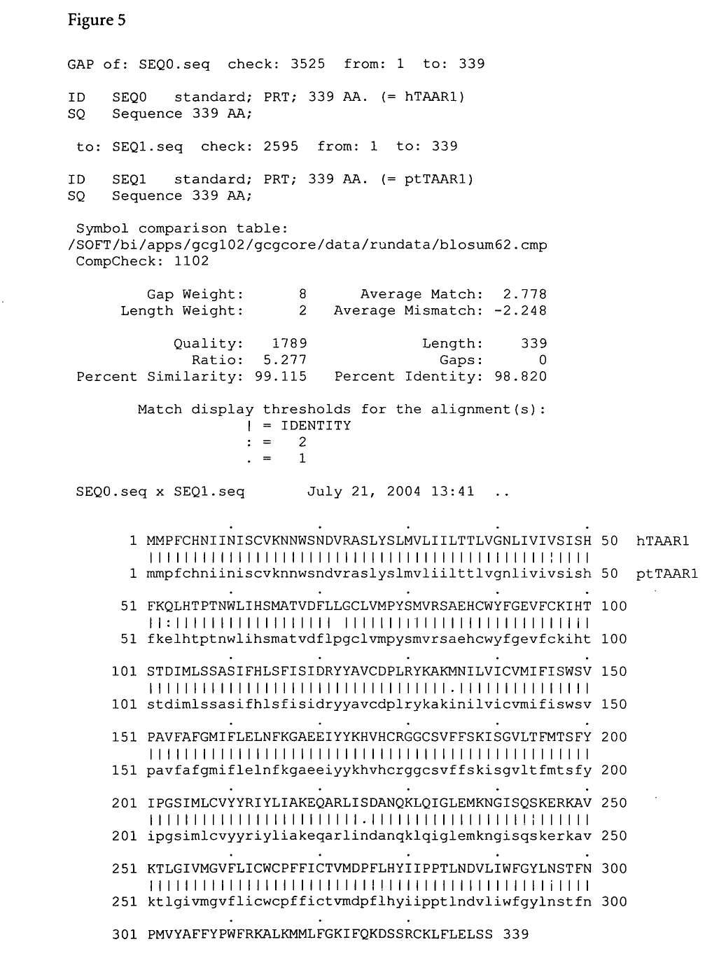

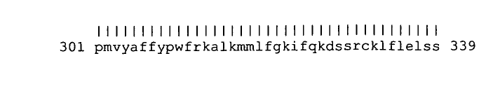

The average divergence between the nucleotide sequences of human and chimpanzee is just 1,2% (Clark et al., Science 2003, 302:1960-1963). The differences between the nucleotide sequences of the functional TAAR of human and chimpanzee is 0.9 to 1.3% (Table 3). The high grade of identity is also found between the amino acid sequences of the chimp TAAR proteins and the equivalent human TAAR proteins (Table 1, FIG. 5). The pharmacology of the chimp TAAR is expected to be the same as of the human TAAR.

TABLE 3
Comparison of human and chimp
TAAR sequences. (ψ = Pseudogene)

| | | | | | Similarity |
|---|---|---|---|---|---|
| Name | | | Chim- | | human/chimpanzee in % |
| Generic | Human | bp | panzee | bp | (DNA: “Identity”) |
| TAAR 1 | TA1 | 1020 | Novel | 1020 | DNA: 99.1; Protein: 98.8 |
| TAAR 2 | novel | 1056 | Novel ψ | 1055 | DNA: 98.4; Protein: 97.2 |
| TAAR 3 | GPR57 ψ | 1030 | Novel ψ | 1030 | DNA: 98.5; Protein: 96.2 |
| TAAR 4 | TA2 ψ, | 1049 | Novel ψ | 1049 | DNA: 98.1; Protein: 96.3 |
| | 5HT-4 ψ | | | | |
| TAAR 5 | PNR | 1014 | Novel | 1014 | DNA: 99.0; Protein: 98.8 |
| TAAR 6 | TA4 | 1038 | Novel | 1038 | DNA: 98.7; Protein: 97.7 |
| TAAR 8 | TA5, | 1029 | Novel ψ | 1027 | DNA: 97.4; Protein: 95.0 |
| | GPR102 | | | | |
| TAAR 9 | TA3 | 1047 | Novel ψ | 1048 | DNA: 97.3; Protein: 95.1 |

The foregoing description illustrates and describes the invention of the present disclosure. Additionally, the disclosure shows and describes only certain embodiments of the invention but, as mentioned above, it is to be understood that the teachings of the present disclosure are capable of use in various other combinations, modifications, and environments and is capable of changes or modifications within the scope of the inventive concept as expressed herein, commensurate with the above teachings and/or the sill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form(s) disclosed herein.

1. A fingerprint sequence specific and selective for chimp trace amine associated receptors (TAARs) wherein the fingerprint sequence comprises an amino acid sequence NSXXNPXXYXXXYXWF (SEQ. ID NO. 1), wherein X is any natural occurring amino acid, N is asparagine, S is serine, P is proline, Y is tyrosine, W is tryptophan and F is phenylalanine.

2. An isolated or recombinant polypeptide comprising the amino acid sequence of SEQ.ID NO.1.

3. A method of identifying mammalian trace amine associated receptors (TAARs) comprising comparing the fingerprint sequence (SEQ.ID NO.1) of claim 1 with a polypeptide sequence, wherein at least a 75% identity of the fingerprint sequence with the polypeptide sequence identifies mammalian trace amine associated receptors (TTARs) within the polypeptide sequence.

4. The method of claim 3, wherein at least a 87% identity of the fingerprint sequence with the polypeptide sequence identifies mammalian trace amine associated receptors (TTARs) within the polypeptide sequence.

5. A method of identifying chimp trace amine receptors (TAARs) comprising comparing the fingerprint sequence (SEQ ID NO. 1) of claim 1 with a polypeptide sequence, wherein a 100% identity of the fingerprint sequence with the polypeptide sequence identifies chimp trace amine associated receptors (TAARs) within the polypeptide sequence.

6. An isolated or recombinant polypeptide comprising SEQ. ID NO. 3 wherein the polypeptide is a trace amine associated receptor (ptTTAR1).

7. An isolated or recombinant polypeptide comprising SEQ. ID NO. 14 wherein the polypeptide is a trace amine associated receptor (ptTAAR5).

8. An isolated or recombinant polypeptide comprising SEQ.ID NO. 16 wherein the polypeptide is a trace amine associated receptor (ptTAAR6).

9. A method for screening and identifying candidate compounds useful in a selected therapy comprising: contacting polypeptide A with a test compound; determining whether said compound binds to the polypeptide A; and determining whether said binding modulates the biological activity of the polypeptide A, wherein the modulation of the biological activity of the polypeptide A by said binding identifies whether said test compound is a candidate compound.

10. The method of claim 9 wherein the selected therapy is selected from one of the group consisting of depression therapy, schizophrenia therapy, migraine therapy, eating disorder therapy and attention-deficit hyperactivity disorder therapy.

11. An isolated or recombinant polynucleotide comprising SEQ ID NO 2 wherein SEQ ID NO. 2 encodes polypeptide ptTAAR1.

12. An isolated or recombinant polynucleotide comprising SEQ ID NO 13 wherein SEQ ID NO. 13 encodes polypeptide ptTAAR15.

13. An isolated or recombinant polynucleotide comprising SEQ ID NO 15 wherein SEQ ID NO. 15 encodes polypeptide ptTAAR6.

14. A vector comprising the polynucleotide selected from the group consisting of SEQ ID NOS 2, 13, or 15.

15. A host cell comprising the vector of claim 14.

16. A transgenic non-human animal comprising a polynucleotide selected from the group consisting of SEQ ID NOS 2, 13 or 15.


SEQUENCE LISTING (46 sequences)

SEQ ID NO: 1 - protein, 16 aa, Mus musculus
  MOD_RES (3)..(4): Variable amino acid
NSXXNPXXYXXXYXWF

SEQ ID NO: 2 - DNA, 1020 bp, Pan troglodytes
  ptTAAR1
atgatgcccttttgccacaatataattaatatttcctgtgtgaaaaacaactggtcaaatgatgtccgtgcttccctgtacagtttaatggtgctcataattctgaccacactcgttggcaatctgatagttattgtttctatatcacacttcaaagaacttcataccccgacaaattggctcattcattccatggccactgtggactttcttccggggtgtctggtcatgccttacagtatggtgagatctgctgagcattgttggtattttggagaagtcttctgtaaaattcacacaagcaccgacattatgctgagctcagcctccattttccatttgtctttcatctccattgaccgctactatgctgtgtgtgatccactgagatataaagccaagatcaatatcttggttatttgtgtgatgatcttcattagttggagtgtccccgctgtttttgcatttggaatgatctttctggagctaaacttcaaaggcgctgaagagatatattacaaacatgttcactgcagaggaggttgctctgtcttctttagcaaaatatctggggtactgacctttatgacttctttttatatacctggatctattatgttatgtgtctattacagaatatatcttatcgctaaagagcaggcaagattaattaatgatgccaatcagaagctccaaattggattggaaatgaaaaatggaatttcacaaagcaaagaaaggaaagctgtgaagacattggggattgtgatgggagttttcctaatatgctggtgccctttctttatctgtacagtcatggacccttttcttcactacattattccacctactttgaatgatgtattgatttggtttggctacttgaactctacatttaatccaatggtttatgcatttttctatccttggtttagaaaagcactgaagatgatgctgtttggtaaaattttccaaaaagattcatccaggtgtaaattatttttggaattgagttcatag

SEQ ID NO: 3 - protein, 339 aa, Pan troglodytes
  ptTAAR1
MMPFCHNIINISCVKNNWSNDVRASLYSLMVLIILTTLVGNLIVIVSISHFKELHTPTNWLIHSMATVDFLPGCLVMPYSMVRSAEHCWYFGEVFCKIHTSTDIMLSSASIFHLSFISIDRYYAVCDPLRYKAKINILVICVMIFISWSVPAVFAFGMIFLELNFKGAEEIYYKHVHCRGGCSVFFSKISGVLTFMTSFYIPGSIMLCVYYRIYLIAKEQARLINDANQKLQIGLEMKNGISQSKERKAVKTLGIVMGVFLICWCPFFICTVMDPFLHYIIPPTLNDVLIWFGYLNSTFNPMVYAFFYPWFRKALKMMLFGKIFQKDSSRCKLFLELSS

SEQ ID NO: 4 - DNA, 1055 bp, Pan troglodytes
  ptTAAR2 Y, pseudogene
atggctgtctcatcagagcaagatgaactttcacatttcaaaagaacacagacaaaaaaggaaacattcaattgctctgaatatggaaatagatcttgcccagaaaatgaaagatctctgggtgtccgagtggctatgtattcatttatggcgggatccatattcatcacaatatttggcaatcttgccgtgataatttccatttcctacttcaagcagcttcacacaccaaccaacttcctcatcctctccatggccatcactgatttcctcctgggattcaccatcatgccatatagtatgatcagatcagtggagaactgctggtattttgggcttacattttgcaagattcattatagttttgatctgatgcttagcataacatccatttttcatctttgctcagtggccattgatagattttatgctgtctgttacccattacgttattccaccaaaataactattccagtcattaaaagattgctacttctatgttggtcggtccctggagcatttgccttcggggtggtcttctcagaggcctatgcagatggaatagagggctatgacatcttggttgcttgttccagttcctgcccagtgatgttcaacaagctatgggggaccaccttgtttatggcaggtttcttcactcctgggtctatgatggtggggatttatggcaaaatttttgcggtatccagaaaacatgctcatgccatcaataacttgcaagaaaatcaaaataatcaagtgaagaaagacaaaaaagctgccaaaactttaggaatagtgataggagttttcttattatgttggtttccttctttcttcacaattttattggatcccttttgaacttctctactcctgtagttttgtttgatgccttgacatggtttggctattttaactccacatgtaatccgttaatatatggtttcttctatccctggtttcgcagagcactgaagtacattttgctaggtaaaattttcagctcatgtttccatactactaatttgtgtatgcaaaaagagagtgagtag

SEQ ID NO: 5 - DNA, 1056 bp, Pan troglodytes
  ptTAAR2 Y f, fixed pseudogene
atggctgtctcatcagagcaagatgaactttcacatttcaaaagaacacagacaaaaaaggaaacattcaattgctctgaatatggaaatagatcttgcccagaaaatgaaagatctctgggtgtccgagtggctatgtattcatttatggcgggatccatattcatcacaatatttggcaatcttgccgtgataatttccatttcctacttcaagcagcttcacacaccaaccaacttcctcatcctctccatggccatcactgatttcctcctgggattcaccatcatgccatatagtatgatcagatcagtggagaactgctggtattttgggcttacattttgcaagattcattatagttttgatctgatgcttagcataacatccatttttcatctttgctcagtggccattgatagattttatgctgtctgttacccattacgttattccaccaaaataactattccagtcattaaaagattgctacttctatgttggtcggtccctggagcatttgccttcggggtggtcttctcagaggcctatgcagatggaatagagggctatgacatcttggttgcttgttccagttcctgcccagtgatgttcaacaagctatgggggaccaccttgtttatggcaggtttcttcactcctgggtctatgatggtggggatttatggcaaaatttttgcggtatccagaaaacatgctcatgccatcaataacttgcaagaaaatcaaaataatcaagtgaagaaagacaaaaaagctgccaaaactttaggaatagtgataggagttttcttattatgttggtttccttctttcttcacaattttattggatccctttttgaacttctctactcctgtagttttgtttgatgccttgacatggtttggctattttaactccacatgtaatccgttaatatatggtttcttctatccctggtttcgcagagcactgaagtacattttgctaggtaaaattttcagctcatgtttccatactactaatttgtgtatgcaaaaagagagtgagtag

SEQ ID NO: 6 - protein, 351 aa, Pan troglodytes
  ptTAAR2 Yf, protein of fixed pseudogene
MAVSSEQDELSHFKRTQTKKETFNCSEYGNRSCPENERSLGVRVAMYSFMAGSIFITIFGNLAVIISISYFKQLHTPTNFLILSMAITDFLLGFTIMPYSMIRSVENCWYFGLTFCKIHYSFDLMLSITSIFHLCSVAIDRFYAVCYPLRYSTKITIPVIKRLLLLCWSVPGAFAFGVVFSEAYADGIEGYDILVACSSSCPVMFNKLWGTTLFMAGFFTPGSMMVGIYGKIFAVSRKHAHAINNLQENQNNQVKKDKKAAKTLGIVIGVFLLCWFPSFFTILLDPFLNFSTPVVLFDALTWFGYFNSTCNPLIYGFFYPWFRRALKYILLGKIFSSCFHTTNLCMQKESE

SEQ ID NO: 7 - DNA, 1030 bp, Pan troglodytes
  ptTAAR3 Y, pseudogene
atggatctaacttatattcccgacgacctatccagttgtccaaaatttgtaaataaatcctgtcctcccaccaaccgctcttttcatgtccgggtgataatgtattcggttatgactggagccatgattatccctattcggaaacttggttataatggtttccatatcgcatttcaaacagcttcactctcccacaaactttctgatcctctccatggcaaccacggactttctgctgggttttgtcattatgccatacaacataatgcgatcagtggagagttgctggtactttggggatggcttttgtaaattccacacaagcttagacatgatgctcagcctgacctccattttccacctctgttccattgctattgaccgattttatgccgtgtgttaccctttacattacacaaccagaacgacaaactccaccataaagcaactgctggcattttgctggtcagttcctgctcttttttcttttggtttagttctatccgaggccaatgtttccggtatgcagagctataagatacttgttgcttgcttcaattactgtgcccttactttcaacaaattctgggggacaatattgttcactacatgtttctttacccctggctccatcatggttggtatttatggcaaaatctttatcgtttccaaacagcatgctcgagtcatcagccatgtgcctgaaaacacaaagggggcagtgaaaaaacacctatccaagaaaaaggacaggaaagcagcgaagacactgggtatagtaatgggggtgtttctggcttgctggttgccttgttttcttgctgttctgattgacccatacctagactactccactcccatactaatattggatcttttagtgtggctcgggtacttcaactctacttgcaaccctcttattcatggctttttttatccatggtttcagaaagcactcaagtacatagtgtcaggaaaaatatttagctcccattcagaaactgcaaatttgtttcctgaagcacattaa

SEQ ID NO: 8 - DNA, 1032 bp, Pan troglodytes
  ptTAAR3 Yf, fixed pseudogene
atggatctaacttatattcccgacgacctatccagttgtccaaaatttgtaaataaatcctgtcctcccaccaaccgctcttttcatgtccgggtgataatgtattcggttatgactggagccatgattatccccatattcggaaacttggttataatggtttccatatcgcatttcaaacagcttcactctcccacaaactttctgatcctctccatggcaaccacggactttctgctgggttttgtcattatgccatacaacataatgcgatcagtggagagttgctggtactttggggatggcttttgtaaattccacacaagcttagacatgatgctcagcctgacctccattttccacctctgttccattgctattgaccgattttatgccgtgtgttaccctttacattacacaaccagaacgacaaactccaccataaagcaactgctggcattttgctggtcagttcctgctcttttttcttttggtttagttctatccgaggccaatgtttccggtatgcagagctataagatacttgttgcttgcttcaattactgtgcccttactttcaacaaattctgggggacaatattgttcactacatgtttctttacccctggctccatcatggttggtatttatggcaaaatctttatcgtttccaaacagcatgctcgagtcatcagccatgtgcctgaaaacacaaagggggcagtgaaaaaacacctatccaagaaaaaggacaggaaagcagcgaagacactgggtatagtaatgggggtgtttctggcttgctggttgccttgttttcttgctgttctgattgacccatacctagactactccactcccatactaatattggatcttttagtgtggctcgggtacttcaactctacttgcaaccctcttattcatggctttttttatccatggtttcagaaagcactcaagtacatagtgtcaggaaaaatatttagctcccattcagaaactgcaaatttgtttcctgaagcacattaa

SEQ ID NO: 9 - protein, 343 aa, Pan troglodytes
  ptTAAR3 Yf, protein of fixed pseudogene
MDLTYIPDDLSSCPKFVNKSCPPTNRSFHVRVIMYSVMTGAMIIPIFGNLVIMVSISHFKQLHSPTNFLILSMATTDFLLGFVIMPYNIMRSVESCWYFGDGFCKFHTSLDMMLSLTSIFHLCSIAIDRFYAVCYPLHYTTRTTNSTIKQLLAFCWSVPALFSFGLVLSEANVSGMQSYKILVACFNYCALTFNKFWGTILFTTCFFTPGSIMVGIYGKIFIVSKQHARVISHVPENTKGAVKKHLSKKKDRKAAKTLGIVMGVFLACWLPCFLAVLIDPYLDYSTPILILDLLVWLGYFNSTCNPLIHGFFYPWFQKALKYIVSGKIFSSHSETANLFPEAH

SEQ ID NO: 10 - DNA, 1049 bp, Pan troglodytes
  ptTAAR4 Y, pseudogene
atgatgaatttgcctgaccctcagaaccccccaacagtacaattttgctttagttcagttaacaattcatgccctagaaatgtgaggccagtgcagagtgtctgggccatgtacctggtcatgatcgggtctatagtgatgacaatgctgggcaacatgatcgtaatgatttccatcgctcacttcaagcagctccactccccgaccaacttcttgatcctctccatggccatcactgactttttgctgagctgtgtggtcatgcccttcagtgtgatcagatccattgagtcctgctggtattttggagacctcttttgcaaagtccacagctgctgtgacatcatgctctgcaccacctccatttttcacctctgcctcatctcagttgaccgttactatgctgtttgcgacccattgcaatatgtcaccagaattaccatccctgtcatagaactctttctactcatcagttggtccattcccatcttttttgcctttggcctggtattctcaaaactaaacataattggtgcagaagagtttgttgcagccattgattgcacaggtttgtgtgtgttaatatttaataagctctgggggggtactggcctcctttatagctttctttctccccgggacaaccatggtgggaatttacatacatatttttacagtagccaggaagcatgccatgcaaattggcacaggttctaggactaaacaggctgggtcaaaaagcaaaaaaaaagtcatcctctaaaacagaaagcaaggccaccaggaccttaggcatagtcatgggagtgtttgtgttgtgctggctgcccttctttgtcttgacgatcacaaatcctttcattaattttacaacccctgaagatctgtacaatgtcttcctctggctgggctatttcaactccgctttcaaccccattttatatggcatgttttatccttggtttcgcaaggcattgaggatgattgtcacaggcatgatcttcctccctgactcttccaccctaagcctgttttctgcccatgcttag

SEQ ID NO: 11 - DNA, 1047 bp, Pan troglodytes
  ptTAAR4 Yf, fixed pseudogene
atgatgaatttgcctgaccctcagaaccccccaacagtacaattttgctttagttcagttaacaattcatgccctagaaatgtgaggccagtgcagagtgtctgggccatgtacctggtcatgatcgggtctatagtgatgacaatgctgggcaacatgatcgtaatgatttccatcgctcacttcaagcagctccactccccgaccaacttcttgatcctctccatggccatcactgactttttgctgagctgtgtggtcatgcccttcagtgtgatcagatccattgagtcctgctggtattttggagacctcttttgcaaagtccacagctgctgtgacatcatgctctgcaccacctccatttttcacctctgcctcatctcagttgaccgttactatgctgtttgcgacccattgcaatatgtcaccagaattaccatccctgtcatagaactctttctactcatcagttggtccattcccatcttttttgcctttggcctggtattctcaaaactaaacataattggtgcagaagagtttgttgcagccattgattgcacaggtttgtgtgtgttaatatttaataagctctggggggtactggcctcctttatagctttctttctccccgggacaaccatggtgggaatttacatacatatttttacagtagccaggaagcatgccatgcaaattggcacaggttctaggactaaacaggctgggtcaaaaagcaaaaaaaagtcatcctctaaaacagaaagcaaggccaccaggaccttaggcatagtcatgggagtgtttgtgttgtgctggctgcccttctttgtcttgacgatcacaaatcctttcattaattttacaacccctgaagatctgtacaatgtcttcctctggctgggctatttcaactccgctttcaaccccattttatatggcatgttttatccttggtttcgcaaggcattgaggatgattgtcacaggcatgatcttcctccctgactcttccaccctaagcctgttttctgcccatgcttag

SEQ ID NO: 12 - protein, 348 aa, Pan troglodytes
  ptTAAR4 Yf, protein of fixed pseudogene
MMNLPDPQNPPTVQFCFSSVNNSCPRNVRPVQSVWAMYLVMIGSIVMTMLGNMIVMISIAHFKQLHSPTNFLILSMAITDFLLSCVVMPFSVIRSIESCWYFGDLFCKVHSCCDIMLCTTSIFHLCLISVDRYYAVCDPLQYVTRITIPVIELFLLISWSIPIFFAFGLVFSKLNIIGAEEFVAAIDCTGLCVLIFNKLWGVLASFIAFFLPGTTMVGIYIHIFTVARKHAMQIGTGSRTKQAGSKSKKKSSSKTESKATRTLGIVMGVFVLCWLPFFVLTITNPFINFTTPEDLYNVFLWLGYFNSAFNPILYGMFYPWFRKALRMIVTGMIFLPDSSTLSLFSAHA

SEQ ID NO: 13 - DNA, 1014 bp, Pan troglodytes
  ptTAAR5
atgagagctgtcttcatccaaggtgctgaagagcaccctgcggcattctgctaccaggtgaatgggtcttgccccaggacagtacatactctgggcatccagttggtcatctacctggcctgtgcagcaggcatgctgattatcgtgctagggaatttatttgtggcatttgctgtgtcctacttcaaagcgcttcacacgcccaccaacttcttgctgctctccctggccctggctgacatgtttctgggtctgctggtgctgcccctcagcaccattcgctcagtggagagctgctggttcttcggggacttcctctgccgcctgcacacctacctggaccccctcttctgcctcacctccatcttccatctctgtttcatttccattgaccgccactgtgccatctgtgaccccctgctctatccctccaagttcacagtgagggtggctctcaggtacatcctggcaggatggggggtgcccgcagcatacacttccttattcctctacacagatgtggtagagacaaggctcagccagtggctggaagagatgccttgtgtgggcagttgccagctgctgctcaataaattttggggctggttaaacttccctttgttctttgtcccctgcctcattatgatcagcttgtatgtgaagatctttgtggttgctaccagacaggctcagcagattaccacattgagcaaaaacctggctggggctgccaagcatgacagaaaagctgccaagaccctgggcattgctgtgggcatatacctcttgtgctggctgcccttcaccatagacacgatggtcgacagcctccttcactttatcacacccccacttgtctttgacatctttatctggtttgcttacttcaactcagcctgcaatcccatcatctatgtcttttcctaccagtggtttcggaaggcactgaaactgacgctgagccagaaggtcttctcaccgcagacacgcactgttgatttgtaccaagaatga

SEQ ID NO: 14 - protein, 337 aa, Pan troglodytes
  ptTAAR5
MRAVFIQGAEEHPAAFCYQVNGSCPRTVHTLGIQLVIYLACAAGMLIIVLGNLFVAFAVSYFKALHTPTNFLLLSLALADMFLGLLVLPLSTIRSVESCWFFGDFLCRLHTYLDPLFCLTSIFHLCFISIDRHCAICDPLLYPSKFTVRVALRYILAGWGVPAAYTSLFLYTDVVETRLSQWLEEMPCVGSCQLLLNKFWGWLNFPLFFVPCLIMISLYVKIFVVATRQAQQITTLSKNLAGAAKHDRKAAKTLGIAVGIYLLCWLPFTIDTMVDSLLHFITPPLVFDIFIWFAYFNSACNPIIYVFSYQWFRKALKLTLSQKVFSPQTRTVDLYQE

SEQ ID NO: 15 - DNA, 1038 bp, Pan troglodytes
  ptTAAR6
atgagcagcaactcatccctgctggtggctgtgcagctgtgctacccgaacgtgaatgggtcctgtgtggaaaccctctactcgcctggatcccgggtgattctgtacatagtgtttggctttggggctgtgttggctgtgtttggaaacctcctggtgatgatttcaatcctccatttcaagcagctgcactctccgaccaattttctcgttgcctctctggcctgcgctgatttcttagtgggtgtgactgtgatgcccttcagcatggtcaggaccgtggagagctgctggtattttgggagaagtttttgtactttccacacctgctgtgatgtggcattttgttactcttctctctttcacttgtgcttcatctccatcgacaggtacattgcggttactgaccccctggtctatcctaccaagttcaccgtatctgtgtcaggaatttgcatcagcgtgtcctggatcctgcccctcatgtacagcggtgctgtgttctacacaggtgtctatgacgatgggctggaggaattatctgatgccctaaactgtataggaggttgtcagaccgttgtaaatcaaaactgggtgttgatagattgtctatccttctttatacctacctttattatgataattctgtatggtaacatatttcttgtggctagacgacaggcgaaaaagatagaaaatactggtagcaagacagaatcatcctcagagagttacaaagccagagtggccaggagagagagaaaagcagctaaaaccctgggggtcacagtggtagcatttatgatttcatggttaccatatagcattgattcattaattgatgcctttatgggctttataacccctgcctatatttatgagatttgctgttggtgtgcttattataactcagccatgaatcctttgatttatgctttattttacccatggtttaggaaagcaataaaagttattgtaactggtcaggttttaaagaacagttcagcaaccatgaatttgttttctgaacatatataa

SEQ ID NO: 16 - protein, 345 aa, Pan troglodytes
  ptTAAR6
MSSNSSLLVAVQLCYPNVNGSCVETLYSPGSRVILYIVFGFGAVLAVFGNLLVMISILHFKQLHSPTNFLVASLACADFLVGVTVMPFSMVRTVESCWYFGRSFCTFHTCCDVAFCYSSLFHLCFISIDRYIAVTDPLVYPTKFTVSVSGICISVSWILPLMYSGAVFYTGVYDDGLEELSDALNCIGGCQTVVNQNWVLIDCLSFFIPTFIMIILYGNIFLVARRQAKKIENTGSKTESSSESYKARVARRERKAAKTLGVTVVAFMISWLPYSIDSLIDAFMGFITPAYIYEICCWCAYYNSAMNPLIYALFYPWFRKAIKVIVTGQVLKNSSATMNLFSEHI

SEQ ID NO: 17 - DNA, 1027 bp, Pan troglodytes
  ptTAAR8 Y, pseudogene
atgaccagcaatttttcctaacctgttgtgcagctttgctatgaggatgtgaatggatcttgtattaaaactccctattctcctgggccctgggtgattctgtacacggcgtttagctttgggtctttgctggctgtatttgcaaatctcttagtaatgacttctgttcttcattttaagcagctgcactctccaaccaattttctcattgcctctctggcctgtgctgacttcttggtaggtgtgactgtgatgcccttcagcatggtcaggacggtggagagctgctggtattttggagccaaattttgtactcttcacagttgctgtgatgtgtcattttgttactcttctgtcctccacttgtgcttcatctgcatcgacaggtacattgcggttactgagcccctggtctatgctaccaagttcaccgtgtccgtgtcgggaatttgcatcagcgtgtgctggattctgcctctcacgtacagcggtgctgtgttctacacaggtgtcaatgatgatgagctgggggagttagtaagtgctctcaactgtgtaggtggctgtcaaattgttgtaagtcaaggctgggtgttgatagattttctgttattcttcatacctacccttgttatgataattctttaccgtaagatttttcttatagctaaacaacaagctataaaaattgaaactactagtagcaaagtagaatcatcctcagagagttataaaatcagagtggccgagagagaggaaagcagctaaaaccctgggggtcacggtactagcatttgttatttcatggttaccgtatacagttgatatattaatcgatgcctttatgggcttcctgacccctgcctatatctatgaaatttgctgttggagtgcttattgtaactcagccatgaatcctttgatttatgctttattttatccttggtttaggaaagccataaaacttattttaagtggggatgttttaaaggctagttcatcagccattagtttattttcagaataa

SEQ ID NO: 18 - DNA, 1029 bp, Pan troglodytes
  ptTAAR8 Yf, fixed pseudogene
atgaccagcaatttttcctatcctgttgtgcagctttgctatgaggatgtgaatggatcttgtattaaaactccctattctcctgggccctgggtgattctgtacacggcgtttagctttgggtctttgctggctgtatttgcaaatctcttagtaatgacttctgttcttcattttaagcagctgcactctccaaccaattttctcattgcctctctggcctgtgctgacttcttggtaggtgtgactgtgatgcccttcagcatggtcaggacggtggagagctgctggtattttggagccaaattttgtactcttcacagttgctgtgatgtgtcattttgttactcttctgtcctccacttgtgcttcatctgcatcgacaggtacattgcggttactgagcccctggtctatgctaccaagttcaccgtgtccgtgtcgggaatttgcatcagcgtgtgctggattctgcctctcacgtacagcggtgctgtgttctacacaggtgtcaatgatgatgagctgggggagttagtaagtgctctcaactgtgtaggtggctgtcaaattgttgtaagtcaaggctgggtgttgatagattttctgttattcttcatacctacccttgttatgataattctttaccgtaagatttttcttatagctaaacaacaagctataaaaattgaaactactagtagcaaagtagaatcatcctcagagagttataaaatcagagtggccaagagagagaggaaagcagctaaaaccctgggggtcacggtactagcatttgttatttcatggttaccgtatacagttgatatattaatcgatgcctttatgggcttcctgacccctgcctatatctatgaaatttgctgttggagtgcttattgtaactcagccatgaatcctttgatttatgctttattttatccttggtttaggaaagccataaaacttattttaagtggggatgttttaaaggctagttcatcagccattagtttattttcagaataa

SEQ ID NO: 19 - protein, 342 aa, Pan troglodytes
  ptTAAR8 Yf, protein of fixed pseudogene
MTSNFSYPVVQLCYEDVNGSCIKTPYSPGPWVILYTAFSFGSLLAVFANLLVMTSVLHFKQLHSPTNFLIASLACADFLVGVTVMPFSMVRTVESCWYFGAKFCTLHSCCDVSFCYSSVLHLCFICIDRYIAVTEPLVYATKFTVSVSGICISVCWILPLTYSGAVFYTGVNDDELGELVSALNCVGGCQIVVSQGWVLIDFLLFFIPTLVMIILYRKIFLIAKQQAIKIETTSSKVESSSESYKIRVAKRERKAAKTLGVTVLAFVISWLPYTVDILIDAFMGFLTPAYIYEICCWSAYCNSAMNPLIYALFYPWFRKAIKLILSGDVLKASSSAISLFSE

SEQ ID NO: 20 - DNA, 1048 bp, Pan troglodytes
  ptTAAR9 Y, pseudogene
atggtgaacaatttctcccaagctgaggctgtggagctgtgttacaagaacgtgaacagatcctgcattaaaactccttactcgccaggtcctcgagctatcctctatgctgtccttggttttggggctgtgctgtcagtgtttggaaacttactggtcatgattgctatccttcacttcaaacaactgcacacacctacaaactttctgatcatgtcgctggcctgtgctgacttcttggtgggagtcactgtgatgcccttcagcacagtgaggtctgtgtagaactgttggtactttggggacagttactgtaaattccatacatgttttgacacatccttctgttttgcttctttatttcatttatgccgtatctctgttgatagatacattgctgttactgatcctctgacctatccaaccaagtttactgtgtcaatttcaggaatatgcattgttctttcctggttcttttctgtcacatacagcttttcgatcttttacacgggagccaacgaagaaggaattgaggaattagtagttgctctaacctgtgtaggaggctgccaggctccactgaatcaaaactgggtcctactttgttttcttctattctttatacccactgtcgctatggtgtttatatacagtaagatatttttggtggccaagcatcaggctaggaagatagaaagtacagccagccaagctcagtcctcctcagagagttacaaggaaagagtagcaaaaagaaagagaaaggctgccaaaaccttgggaattgctatggcagcatttcttgtctcttggctaccatacatcgttgatgcagtgattgatgcttacatgaattttataactcctccttatgtttatgagattttagtttggtgtgtttattataattcagctatgaaccccttgatttatgctttcttttacccatggtttcggaaggcaataaaacttatttgtaagcggcaaggtcttaaggagtgattcatcaacaactaatttattttctgaagaagtagagaaagatcaa

SEQ ID NO: 21 - DNA, 1047 bp, Pan troglodytes
  ptTAAR9 Yf, fixed pseudogene
atggtgaacaatttctcccaagctgaggctgtggagctgtgttacaagaacgtgaacagatcctgcattaaaactccttactcgccaggtcctcgagctatcctctatgctgtccttggttttggggctgtgctgtcagtgtttggaaacttactggtcatgattgctatccttcacttcaaacaactgcacacacctacaaactttctgatcatgtcgctggcctgtgctgacttcttggtgggagtcactgtgatgcccttcagcacagtgaggtctgtgtacaactgttggtactttggggacagttactgtaaattccatacatgttttgacacatccttctgttttgcttctttatttcatttatgccgtatctctgttgatagatacattgctgttactgatcctctgacctatccaaccaagtttactgtgtcaatttcaggaatatgcattgttctttcctggttcttttctgtcacatacagcttttcgatcttttacacgggagccaacgaagaaggaattgaggaattagtagttgctctaacctgtgtaggaggctgccaggctccactgaatcaaaactgggtcctactttgttttcttctattctttatacccactgtcgctatggtgtttatatacagtaagatatttttggtggccaagcatcaggctaggaagatagaaagtacagccagccaagctcagtcctcctcagagagttacaaggaaagagtagcaaaaagaaagagaaaggctgccaaaaccttgggaattgctatggcagcatttcttgtctcttggctaccatacatcgttgatgcagtgattgatgcttacatgaattttataactcctccttatgtttatgagattttagtttggtgtgtttattataattcagctatgaaccccttgatttatgctttcttttacccatggtttcggaaggcaataaaacttattgtaagcggcaaggtcttaaggagtgattcatcaacaactaatttattttctgaagaagtagagaaagatcaa

SEQ ID NO: 22 - protein, 349 aa, Pan troglodytes
  ptTAAR9 Yf, protein of fixed pseudogene
MVNNFSQAEAVELCYKNVNRSCIKTPYSPGPRAILYAVLGFGAVLSVFGNLLVMIAILHFKQLHTPTNFLIMSLACADFLVGVTVMPFSTVRSVYNCWYFGDSYCKFHTCFDTSFCFASLFHLCRISVDRYIAVTDPLTYPTKFTVSISGICIVLSWFFSVTYSFSIFYTGANEEGIEELVVALTCVGGCQAPLNQNWVLLCFLLFFIPTVAMVFIYSKIFLVAKHQARKIESTASQAQSSSESYKERVAKRKRKAAKTLGIAMAAFLVSWLPYIVDAVIDAYMNFITPPYVYEILVWCVYYNSAMNPLIYAFFYPWFRKAIKLIVSGKVLRSDSSTTNLFSEEVEKDQ

SEQ ID NO: 23 - DNA, 18 bp, Pan troglodytes
  schTAR1_5_01, 5′ primer
atgatgcccttttgccac

SEQ ID NO: 24 - DNA, 27 bp, Pan troglodytes
  schTAR1_3_01, 3′ primer
ctatgaactcaattccaaaaataattt

SEQ ID NO: 25 - DNA, 25 bp, Pan troglodytes
  schGPR58_5_01, 5′ primer
gaaacattcaattgctctgaatatg

SEQ ID NO: 26 - DNA, 21 bp, Homo sapiens
  schGPR58_3_02, 3′ primer
tgcttcaatttattcatgcag

SEQ ID NO: 27 - DNA, 21 bp, Pan troglodytes
  chGPR58_ex1_5_02, 5′ primer
ctaaggagcctgatctcaacc

SEQ ID NO: 28 - DNA, 20 bp, Pan troglodytes
  chGPR58_ex1_3_01, 3′ primer
gctctgtgtgatctccgttg

SEQ ID NO: 29 - DNA, 19 bp, Homo sapiens
  schGPR57_5_02, 5′ primer
cagtgactcatcctcctgg

SEQ ID NO: 30 - DNA, 21 bp, Homo sapiens
  schGPR57_3_02, 3′ primer
tctattcacttttgcaacagc

SEQ ID NO: 31 - DNA, 20 bp, Pan troglodytes
  schTAR2_5_01, 5′ primer
atgatgaatttgcctgaccc

SEQ ID NO: 32 - DNA, 21 bp, Pan troglodytes
  schTAR2_3_01, 3′ primer
ctaagcatgggcagaaaacag

SEQ ID NO: 33 - DNA, 22 bp, Pan troglodytes
  schPNR_5_01, 5′ primer
atgagagctgtcttcatccaag

SEQ ID NO: 34 - DNA, 23 bp, Pan troglodytes
  schPNR_3_01, 3′ primer
tcattcttggtacaaatcaacag

SEQ ID NO: 35 - DNA, 24 bp, Pan troglodytes
  schTAR4_5_02, 5′ primer
cttctccatatgtaaataacagcg

SEQ ID NO: 36 - DNA, 25 bp, Pan troglodytes
  schTAR4_3_02, 3′ primer
gtatcctgaacttcgtctatacaac

SEQ ID NO: 37 - DNA, 22 bp, Homo sapiens
  schTAR5_5_01, 5′ primer
atgaccagcaatttttcctaac

SEQ ID NO: 38 - DNA, 26 bp, Homo sapiens
  schTAR5_3_01, 3′ primer
ttattctgaaaataaactaatggctg

SEQ ID NO: 39 - DNA, 19 bp, Pan troglodytes
  schTAR3_5_01, 5′ primer
atggtgaacaatttctccc

SEQ ID NO: 40 - DNA, 27 bp, Pan troglodytes
  schTAR3_3_01, 3′ primer
ttaatctttctctacttcttcagaaaa

SEQ ID NO: 41 - DNA, 1032 bp, Homo sapiens
  CDS (1)..(1029)
atggatctaacttatattcccgaagacctatccagttgtccaaaatttgtaaataaatcctgtcctcccaccaaccgctcttttcatgtccaggtgataatgtattcggttatgactggagccatgattatcccactattcggaaacttggttataatggtttccatatcgcatttcaaacagcttcactctcccacaaactttctgatcctctccatggcaaccacggactttctgctgggttttgtcattatgccatacagcataatgcgatcagtggagagttgctggtactttggggatggcttttgtaaattccacacaagctttgacatgatgctcagactgacctccattttccacctctgttccattgctattgaccgattttatgccgtgtgttaccctttacattacacaaccaaaatgacgaactccaccataaagcaactgctggcattttgctggtcagttcctgctcttttttcttttggtttagttctatctgaggccgatgtttccggtatgcagagctataagatacttgttgcttgcttcaatttctgtgcccttactttcaacaaattctgggggacaatattgttcactacatgtttctttacccctggctccatcatggttggtatttatggcaaaatctttatcgtttccaaacagcatgctcgagtcatcagccatgtgcctgaaaacacaaagggggcagtgaaaaaacacctatccaagaaaaaggacaggaaagcagcgaagacactgggtatagtaatgggggtgtttctggcttgctggttgccttgttttcttgctgttctgattgacccatacctagactactccactcccatactaatattggatcttttagtgtggctccggtacttcaactctacttgcaaccctcttattcatggcttttttaatccatggtttcagaaagcattcaagtacatagtgtcaggaaaaatatttagctcccattcagaaactgcaaatttgtttcctgaagcacattaa

SEQ ID NO: 42 - protein, 343 aa, Homo sapiens
MDLTYIPEDLSSCPKFVNKSCPPTNRSFHVQVIMYSVMTGAMIIPLFGNLVIMVSISHFKQLHSPTNFLILSMATTDFLLGFVIMPYSIMRSVESCWYFGDGFCKFHTSFDMMLRLTSIFHLCSIAIDRFYAVCYPLHYTTKMTNSTIKQLLAFCWSVPALFSFGLVLSEADVSGMQSYKILVACFNFCALTFNKFWGTILFTTCFFTPGSIMVGIYGKIFIVSKQHARVISHVPENTKGAVKKHLSKKKDRKAAKTLGIVMGVFLACWLPCFLAVLIDPYLDYSTPILILDLLVWLRYFNSTCNPLIHGFFNPWFQKAFKYIVSGKIFSSHSETANLFPEAH

SEQ ID NO: 43 - protein, 35 aa, Pan troglodytes
LMMSHTDILSSIFTFSFGFWFFTLIWGYVGGCSVF

SEQ ID NO: 44 - protein, 35 aa, Pan troglodytes
ACLSHTDPFCLIYLNLFCYWFTTFIWAYMGSCQLL

SEQ ID NO: 45 - protein, 35 aa, Pan troglodytes
VFMSHTDVFCYLYIDSFTFWYSSCCWAYLGGCQTV

SEQ ID NO: 46 - protein, 339 aa, Homo sapiens
MMPFCHNIINISCVKNNWSNDVRASLYSLMVLIILTTLVGNLIVIVSISHFKQLHTPTNWLIHSMATVDFLLGCLVMPYSMVRSAEHCWYFGEVFCKIHTSTDIMLSSASIFHLSFISIDRYYAVCDPLRYKAKMNILVICVMIFISWSVPAVFAFGMIFLELNFKGAEEIYYKHVHCRGGCSVFFSKISGVLTFMTSFYIPGSIMLCVYYRIYLIAKEQARLISDANQKLQIGLEMKNGISQSKERKAVKTLGIVMGVFLICWCPFFICTVMDPFLHYIIPPTLNDVLIWFGYLNSTFNPMVYAFFYPWFRKALKMMLFGKIFQKDSSRCKLFLELSS